(19) United States
(12) Patent Application Publication
(10) Pub. No.: US 2010/0022811 A1
(43) Pub. Date: Jan. 28, 2010

(54) SELECTIVE CATALYTIC CRACKING PROCESS OF NATURAL GAS LIQUID FRACTION TO LIGHT OLEFINS AND OTHER PRODUCTS

(75) Inventors: Eduardo Falabella SOUSA AGUIAR, Rio de Janeiro (BR); Cláudio José DE ARAUJO MOTA, Rio de Janeiro (BR); José Luiz FONTES MONTEIRO, Rio de Janeiro (BR); Débora Prado BATISTA PEIXOTO, Rio de Janeiro (BR); Adriano DO COUTO FRAGA, Rio de Janeiro (BR); Ana Carlota BELIZARIO DOS SANTOS, Rio de Janeiro (BR); Antonio Marcos FONSECA BIDART, Rio de Janeiro (BR); Fernando Barbosa DE OLIVEIRA, Rio de Janeiro (BR); Henrique Soares CERQUEIRA, Rio de Janeiro (BR); Isabela Brito DOS SANTOS, Rio de Janeiro (BR); Marcos DE FREITAS SUGAYA, Rio de Janeiro (BR)

Correspondence Address: NIXON & VANDERHYE, PC, 901 NORTH GLEBE ROAD, 11TH FLOOR, ARLINGTON, VA, 22203, US

(73) Assignee: PETROLEO BRASILEIRO S.A. - PETROBRAS, Rio de Janeiro (BR)

(21) Appl. No.: 12/572,575

(22) Filed: Oct. 2, 2009

Related U.S. Application Data
(63) Continuation of application No. 11/498,261, filed on Aug. 3, 2006.

(30) Foreign Application Priority Data
Jun. 1, 2005 (BR) PI 0502015-8

(51) Int. Cl. C07C 15/00; C07C 4/02

(52) U.S. Cl. 585/415; 585/653; 585/651

(57) ABSTRACT

A selective catalytic cracking process of natural gas liquid fraction to light olefins and other products is described, the process includes placing in contact (within a reaction zone) said liquid fraction of natural gas, rich in C₅⁺ paraffins, with an MFI type zeolitic catalyst in acid form, having a pore size of at least 4 Angstroms, a silica/alumina ratio of between 10 and 2000, and where the processing conditions involve a temperature of between 350° C. and 650° C., space velocity of between 2 and 100h⁻¹ and atmospheric pressure, and afterwards carrying out the catalytic cracking to separate products, to recover a product enriched with light olefins, LPG fractions, and aromatics, and where the production of olefins is favored in conditions of higher space velocities, while the production of LPG fractions and aromatics are favored in conditions of lowered space velocities.

### SCOPE OF THE INVENTION

The present invention belongs in the field of the catalytic cracking processes, more particularly, of the catalytic cracking of condensed natural gas fraction (C₅⁺) to obtain light olefins and the other petrochemicals, such as the GLP fraction and aromatics. The process is carried out in the presence of an MFI type zeolitic catalyst in acid form.

### FUNDAMENTALS OF THE INVENTION

The increasing necessity to preserve the environment as well as the search for fuels with lower particulate emission and gaseous pollutants such as nitrogen oxides (NOx), carbon dioxide (CO₂) and, chiefly, carbon monoxide (CO), increases the demand for light olefins, which are used in the alkylation processes to obtain higher octane fuel and in the synthesis processes of oxygenated fuels, such as MTBE, ETBE and DME, as mentioned in articles by Buchanan, J. S., Catalysis, Today, 55, (2000), 207-212, J. H Lunsford, Catalysis Today, 2000, 63, 165 and in Ullmann's, Encyclopedia of Industrial Chemistry, VA 8, ed. VCH., p. 541.

Moreover, the polymerization processes used to manufacture thermoplastics such as polyethylene and polypropylene, which use ethane and propane, respectively, as raw material, also contribute to the increase of demand for light olefins.

These basic petrochemical raw materials are traditionally produced by cracking naphtha. However, the strong growth in the world-wide demand for these raw materials that occurred between 1990-1997, made the search for new production methods extremely important, especially for propane, the generation of which (by cracking naphtha) is less than the consumption by petrochemical industries. Forecasts carried out by the Brazilian Association of Chemical Industries (ABIQUIM) indicate that, in Brazil, the offering of light olefins produced by refining naphtha fractions will begin to be lower than the demand, from an optimistic viewpoint, starting in the year 2006, in accordance with the ABIQUIM Economic Commission's Annual Report: “Demand for Petrochemical Raw Materials and Probable Origin up until 2010”, Otto V. Perrone, Carlos A. Daccache, Leonidas C. de M. Filho, Lucy H. M. N. Santos, Marcelo Wasem and Suzana Tintner, December/2002.

The world-wide offering of natural gas is not only increasing due to the decline of petroleum reserves, but also due to the fact that existing natural gas reserves are not concentrated in the Middle East, as in the case of petroleum.

Natural gas has occupied an ever increasing position of importance and with a view towards an increase in demand during the next decades of the 21^{st} century. As a consequence of fluctuations in price on the world naphtha market, the search for alternative routes using natural gas to obtain petrochemical supplies has been increasing.

When natural gas arrives at the Natural Gas Processing Units (NGPU), it undergoes compression or absorption processes, through which, at the end, processed natural gas fractions (C₁-C₂), LPG (C₃-C₄) are obtained, as well as condensed natural gas fractions (C₅⁺), also known as natural gasoline. The LPG is basically used as residential gas, according to Moutinho dos Santos, E.; Zamalloa, G. C.; Villanueva, L. Dondero; Faga.; M. T. Werneck; “Natural Gas: Strategies for New Energy in Brazil”, ANNABLUME Publishing Company, 1^{st} edition, August/2002.

Natural gas generates 60% of the LPG produced in the world, while, in Brazil, this percentage is only 15%, according to the Annual Report of the BNDES Chemical Complex: Natural Gas as Raw Material for the Production of Ethene in the State of Rio de Janeiro”, Montenegro, R. S. P. and Koo Pan, S. S, 2000.

The C₅⁺ fraction is rich in saturated hydrocarbons such as pentanes, hexanes and heptanes and is of low commercial value. Normally, it is incorporated into gasoline to adjust the vapor pressure of same while at the same time it is mixed into crude oil to facilitate pipeline draining. With the increase in the use and production of natural gas, this fraction has become extremely important from the business point of view.

Zeolitic materials, both natural and synthetic, are known for their catalytic properties in reactions with hydrocarbons, where they are used, for example, in cracking, hydrocracking, hydroisomerization, methanol production of gasoline, xylene isomerization, ethylbenzene synthesis, hydrocarbon oxidation processes, etc. See the article on this subject by Cusumano, J., Perspectives in Catalysis, J. M. Thomas and K. Zamarev (eds.), IUPAC, Chemistry for 21st Century, Blackwell Scientific Publication, 1992.

Typically, zeolites are porous crystalline aluminosilicates having a defined structure with cavities interconnected by channels. The dimensions of the cavities and channels in the structure of these materials are within a range of 3 to 13 Å. These are typical diameter measurements of organic molecules, which allow selective separation of hydrocarbons, by using those known as molecular sieves, in accordance with the publication by Breck, D. W. and Krieger, Robert E., Molecular Zeolite Sieves, Publishing Company, Malabar, Flowery (1984).Zeolites are classified by the type of topology its crystalline grid presents, without taking in consideration its composition, the distribution of the various tetrahedral atoms, the dimensions of unitary cells and symmetry.

One type of zeolite that is often utilized in catalytic cracking processes of gas oil, (as in the article mentioned above by Buchanan, J. S., Catalysis, Today, 55, (2000), 207-212), and in the production of olefins from methanol, (see article by Olah, G. and Molnar A., Hydrocarbon Chemistry, ed, John Wiley & Sons, Ina, 1995, P. 88) is the MFI type, a crystalline solid with a structure that includes pores of an average diameter ranging between 4 and 7 Å. The opening of the pores contains 10 atoms of oxygen.

One MFI zeolite that is quite well known for its high stability and for presenting low coke formation is ZSM-5. See the article on this subject by H. van Koningsveld, J. C. Jansen and H. van Bekkum, Zeolites, (1990), 10, 235.

Many publications mention the use of zeolites of the MFI type, in isomerization reactions from linear olefins C₂-C₁₀ to olefinic products C₄-C₇, such as for example, in patent EP-A-0026041.

Other patents, such as for example, U.S. Pat. Nos. 6,118,035, 6,566,293B1, and international publication WO 01/64761A2, already mention the use of MFI zeolites as additives in the FCC catalytic cracking process in a fluidized bed (Fluid Catalytic Cracking). See also the article by Buchanan, J. S., mentioned above, where increasing the reaction temperature favors an increase in selectivity of light olefins.

However, in another specific type of cracking process, known as DCC (Deep Catalytic Cracking), it is necessary to increase the temperature and the vapor/oil ratio. However, thermal cracking is not very selective, in which the formation of large amounts of products of lower commercial value occur, like hydrogen, methane and ethane, as in Patent U.S. Pat. No. 6,566,293 B1. The use of MFI type zeolites as conventional additives is also mentioned in some patents that normally include a source of phosphorous, as in the European patent application EP-A-511013 and international publication WO 94/13754.

In other patent documents, the use of MFI type zeolites as additives when mixed with zeolite Y is mentioned, as in U.S. Pat. No. 5,472,594, to obtain products with a high level of C₄-C₅ olefins or used as an additive mixed to kaolin and phosphoric acid, as in U.S. Pat. No. 5,521,133. However, the additives dilute the catalyst and lead to a decrease in conversion, as in U.S. Pat. No. 5,521,133.

It is well known that reaction models with n-hexane and 3-methyl-pentane show that the acid zeolites frequently have an important role in industrial processes of paraffin catalytic cracking which are frequently used as reaction models in studies of the properties of zeolite acids, as according to the publications of P. Voog and H. Van Bekkum, Applied Catalysis, 59, 1991, 3311-331 and of John Abbot, Applied Catalysis, 57, 1990, 105-125.

Therefore, in spite of existing developments, the technique still needs a process of selective catalytic cracking on a MFI type zeolitic catalyst where the load is the liquid C₅⁺fraction of condensed natural gas made up of mostly paraffins, to obtain light olefins and other petrochemical supplies such as the LPG fraction and aromatics. Said catalytic cracking process is described and claimed in the present application.

### SUMMARY OF THE INVENTION

In a broad sense, the process of selective catalytic cracking of the liquid fraction of natural gas to light olefins and other products in accordance with the invention includes placing said liquid fraction of natural gas, rich in C5+ paraffins, in contact (within a reaction zone) with an MFI type zeolitic catalyst in acid form, having a pore size of at least 4 Angstroms, a silica/alumina ratio of between 10 and 2000, and where the processing conditions involve a temperature of between 350° C. and 650° C., space velocity of between 2 and 100h⁻¹ at atmospheric pressure, and afterwards carrying out the catalytic cracking to separate products, to recover a product enriched with light olefins, LPG fractions and aromatics, and where the production of olefins is favored in conditions of higher space velocities, while the production of LPG fractions and aromatics are favored in conditions of lowered space velocities.

Thus, the invention proves a selective catalytic cracking process of condensed natural gas fraction to obtain light olefins, LPG and aromatics.

Moreover, the invention proves a selective catalytic cracking process of condensed natural gas fraction to obtain light olefins, LPG and aromatics in the presence of an MFI type catalyst.

The invention also proves an MFI type catalyst to be used in said selective catalytic cracking process.

### DETAILED DESCRIPTION OF THE PREFERRED EMBODIMENTS

One aspect of the invention deals with a selective catalytic cracking process of the condensed fraction of natural gas, rich in C₅⁺ paraffins, in the presence of an MFI type catalyst in acid form, with a silica/alumina ratio of between 10 and 2000, preferably of between 50 and 500, even more preferably of between 20 and 100, at a temperature of between 350° C. and 600° C., preferably at 530° C., and at a space velocity of between 2 and 100h⁻¹, and afterwards to carry out the cracking, to recover a product enriched with light olefins, LPG fractions, and aromatics.

The useful load for the process of the invention is a condensed natural gas rich in C₅⁺ paraffins. Typical compositions of the condensed load are listed in the Tables corresponding to Examples 1, 2, and 3 below.

As is described, an example of the invention of a catalytic cracking process is performed at laboratory scale. The conditions adopted for space velocity (WHSV) are those used for natural condensed gas load injection at atmospheric pressure. Catalytic tests have a duration of approximately 24 hours.

The reaction temperature applied during our catalytic tests was preferably at 530° C., but the process is equally operational at a higher range of temperature, between 350° C. and 650° C.

The catalytic test unit is coupled with a gas chromatographer equipped with a Plot KCl/Al₂O₃ capillary column with 50m to identify the products.

The load of natural condensed gas is maintained with a continuous stream on a catalyst mass for a period of approximately 24 hours.

The operational conditions used in the catalytic tests are described in our Examples 1, 2, and 3.

In another aspect, the invention deals with a useful MFI catalyst in the present process of selective catalytic cracking.

Thus, zeolites with an average pore size that is useful for the invention are described in “Atlas of Zeolite Structure Types”, eds. W. H. Meier and D. H. Olson, Butterworth-Heineman, Third Edition, 1992.

Zeolites with an average pore size exhibit a pore size of between 5 and 7 Angstroms and include, for example, zeolites with an MFI, MFS, MEL, MTW, EUO, MTT, HEU, FER, and TON structure. Non-restrictive examples of these zeolites of average pore size include ZSM-5, ZSM-12, ZSM-22, ZSM-23, ZSM-34, ZSM-35, ZSM-38, ZSM-48, ZSM-50, silicalite, and silicalite 2. Other zeolites of average pore size include the Silico-Aluminophosphate (SAPO), as SAPO-4 and SAPO-11, Chromosilicates, Gallium Silicates, Silicates of Iron; Aluminum Phosphates (ALPO), as ALPO-11, Titanium Aluminosilicates (TASO), as TASO-45; Boron Silicates; Aluminophosphate of Titanium (TAPO), as TAPO-11; and Aluminosilicates of Iron.

An especially useful catalyst for the practice of the invention exhibits a zeolitic structure of the MFI type (called MFI), preferably containing a SiO₂/Al₂O₃ molar ratio of between 20 and 2000. The textural properties and the chemical analysis of this typical catalyst, are presented in Table 1, through the area specific (BET) and superficial results obtained. Additionally, the micropore and mesopore volume is determined by nitrogen adsorption at −196° C. (T-Plot Method). The chemical analysis is determined by X-Ray fluorescence. From this analysis the SiO₂/Al₂O₃ molar ratio is calculated. A preferred MFI zeolite catalyst is the ZSM-5.

Although the following Examples have been produced using a specific catalyst, it should be quite clear to specialists that other catalysts of the same type may be used.

Still, although the tests here reported will be performed using a pure zeolite catalyst, on an industrial scale, it will be made up in the usual way with matrix, binder, etc.

Before using the catalyst, it is pre-macerated and sifted, having been acquired by grain size distribution between 0.510 mm and 0.044 mm (20 and 250 Tyler mesh), preferably between 0.247 mm and 0.091 mm (42 and 115 Tyler mesh) to lower the incidence of any problems relative to diffusion control during the catalytic tests. On an industrial scale, the catalyst is used in the form of dust, pellet or any other configuration.

The catalyst is pretreated at a temperature within the range of between 200-650° C., preferably between 300-600° C., more specifically, 530° C., for a period of 2 hours with a heating rate of 5° C./min under the continuous flow of a carrier gas (preferably air or nitrogen) at 40 ml/min.

TABLE 1
Textural Analysis

| | |
|---|---|
| Specific Area (m²/g) | 422 |
| Micropore Volume (cm³/g) | 0.173 |
| Mesopore Volume (cm³/g) | 0.098 |
| External Area (m²/g) | 53 |

Chemical Analysis (% p/p)

| | |
|---|---|
| SiO₂ | 97.97 |
| Al₂O₃ | 2.035 |
| Na₂O | <0.005 |
| SiO₂/Al₂O₃ molar ratio of the grid | 82 |

The textural properties and chemical composition of a catalyst adjusted for the process of the invention are listed in Table 1.

The performance of the catalyst used in different operational conditions was evaluated through the effluent's composition values (% weight) and from the calculations of selectivity (% weight) calculated from the results obtained through the conventional chromatography technique in the gaseous phase.

The terms used in the Example tables and the calculation of selectivity (% weight) are defined below:

Total Olefins=Ethene (% weight)+Propene (% weight)+Butenes (% weight) 1)

LPG Fraction=Propane (% weight)+Butanes (% weight) 2)

Total Aromatics (%)=Benzene (% weight)+Ethylbenzene (% weight)+Toluene (% weight)+p and m-Xylene (% weight)+o-Xylene (% weight)+1,2,3 Trimethyl Benzene (% weight)+1,2,4 Trimethyl Benzene (% weight)+1,3,5 Trimethyl Benzene (% weight) 3)

$$Selectivity of products ( \% p ) = \frac{[ \begin{matrix}Composition of the \\ Product ( \% weight ) \times \\ 100\end{matrix} ]}{Load Conversion} \qquad 4 )$$

The invention will be illustrated by the following Examples, which should not be considered restrictive.

In Example Tables 2, 3, and 4 show the results of the composition (% weight) of the effluent for the catalytic tests or Examples 1, 2, and 3, respectively, after 2, 12 and 24 hours of reaction.

Tables 5, 6, and 7 show the results of product selectivity (% weight) obtained for these same reaction times, respectively, for the tests or Examples 1, 2, and 3.

### EXAMPLE 1

The catalyst used is an MFI type zeolite (called MFI). The catalyst is pre-macerated and sifted, having been acquired by grain size distribution between 0.247 mm and 0.091 mm (42 and 115 Tyler mesh). It is pretreated at a temperature of 530° C. for 2 hours with a heating rate of 5° C./min under a continuous flow at 40 ml/min, preferably with synthetic air. The reaction takes place at a temperature of 530° C.

The load of natural condensed gas is maintained with a continuous stream at 0.599 ml/min on a catalyst mass of 0.2626 g (WHSV=85.10h⁻¹), for a period of approximately 24 hours.

The performance of the catalyst is evaluated for the reaction times corresponding to 2, 12, and 24 hours that are shown in Table 2 through the result of the effluent composition (% weight) for the test in Example 1.

The calculated selectivity values (% weight) for these same reaction times are shown compiled in Table 5.

TABLE 2

Composition (% Weight)

| Compounds | Feed | 2 H | 12 H | 24 H |
|---|---|---|---|---|
| Methane | — | 0.3 | 0.3 | 0.2 |
| Ethane | — | 0.6 | 0.7 | 0.6 |
| Ethene | — | 1.3 | 1.5 | 1.2 |
| Propane | — | 2.4 | 2.7 | 2.1 |
| Propene | — | 2.9 | 3.6 | 3.1 |
| Isobutane | 0.2 | 0.6 | 0.6 | 0.4 |
| n-Butane | 9.9 | 11.2 | 8.2 | 9.3 |
| trans-2-Butene | — | 0.5 | 0.6 | 0.6 |
| 1-Butene | — | 0.4 | 0.5 | 0.4 |
| Isobutane | — | 0.9 | 1.1 | 1.0 |
| cis-2-Butene | — | 0.4 | 0.5 | 0.4 |
| Isopentane | 24.6 | 23.2 | 21.0 | 21.3 |
| n-Pentane | 28.2 | 25.3 | 24.2 | 24.0 |
| Pentanes | — | — | — | — |
| C6 Sat + Insat | 25.6 | 20.4 | 23.4 | 23.2 |
| C7 Sat + Insat | 8.2 | 6.2 | 7.1 | 8.8 |
| C8 Sat + Insat | 2.1 | 1.7 | 1.6 | 1.6 |
| C9 Sat + Insat | 0.2 | 0.2 | — | — |
| Benzene | 0.5 | 0.6 | 0.6 | 0.5 |
| Toluene | 0.4 | 0.9 | 1.0 | 0.8 |
| Ethyl-Benzene | — | — | 0.1 | 0.1 |
| p and m-Xylene | 0.1 | — | 0.5 | 0.3 |
| o-Xylene | — | — | 0.1 | 0.1 |
| 1,2,3 Trimethylbenzene | — | — | — | — |
| 1,2,4 Trimethylbenzene | — | — | — | — |
| 1,2,3 Trimethylbenzene | — | — | — | — |
| Others | — | — | — | — |
| Totals Olefins (%) (C2-C4) | — | 6.4 | 7.8 | 6.7 |
| LPG Fraction (%) | 10.1 | 14.2 | 11.5 | 11.8 |
| Total Aromatics (%) | 1.0 | 1.5 | 2.4 | 1.8 |
| Load Conversion (%) | | 11.5 | 10.9 | 9.4 |

H = hours;
WHSV = 85.1 h⁻¹;
Reaction Temperature = 530° C.

### EXAMPLE 2

The catalyst used is an MFI type zeolite (called MFI). The catalyst is pre-macerated and sifted, having been acquired by grain size distribution between 0.247 mm and 0.091 mm (42 and 115 Tyler mesh). It was pretreated at a temperature of 530° C. for 2 hours with a heating rate of 5° C./min under the continuous flow at 40 ml/min, preferably with synthetic air. The reaction takes place at a temperature of 530° C.

The load of natural condensed gas is maintained with a continuous stream at 0.3020 ml/min on a catalyst mass of 0.5020 g (WHSV=22.44h⁻¹), for a period of approximately 24 hours.

Table 3 shows the result of the effluent composition (% weight) for the test in Example 2, in which the performance of the catalyst is evaluated for the reaction times corresponding to 2, 12 and 24 hours. The calculated selectivity values (% weight) for these same reaction times are shown in Table 6.

### EXAMPLE 3

The catalyst used is an MFI type zeolite (called MFI). The catalyst is pre-macerated and sifted, having been acquired by grain size distribution between 0.247 mm and 0.091 mm (42 and 115 Tyler mesh). It was pretreated at a temperature of 530 ° C. for 2 hours with a heating rate of 5° C./min under the continuous flow at 40 ml/min, preferably with synthetic air. The load of natural condensed gas is maintained with a continuous stream at 0.1425 ml/min on a catalyst mass of 1.0133 g (WHSV=5.25h⁻¹), for a period of approximately 24 hours. The performance of the catalyst is evaluated for the reaction times corresponding to 2, 12, and 24 hours.

The results of the effluent composition (% weight) for Test 3 are shown in Table 4.

The calculated selectivity values (% weight) for these same reaction times are shown in Table 7.

TABLE 3

Composition (% Weight)

| Compounds | Feed | 2 H | 12 H | 24 H |
|---|---|---|---|---|
| Methane | — | 1.1 | 1.1 | 0.9 |
| Ethane | — | 2.7 | 2.7 | 2.2 |
| Ethene | — | 3.8 | 3.6 | 3.2 |
| Propane | — | 10.7 | 9.7 | 7.3 |
| Propene | — | 7.2 | 7.0 | 6.3 |
| Isobutane | 0.1 | 2.4 | 2.1 | 1.5 |
| n-Butane | 16.8 | 10.9 | 10.6 | 11.7 |
| trans-2-Butene | — | 1.2 | 1.2 | 1.1 |
| 1-Butene | — | 0.9 | 0.9 | 0.8 |
| Isobutane | — | 2.1 | 2.0 | 1.8 |
| cis-2-Butene | — | 0.9 | 0.9 | 0.8 |
| Isopentane | 30.7 | 16.3 | 16.5 | 19.0 |
| n-Pentane | 32.3 | 14.7 | 15.3 | 17.8 |
| Pentenes | — | 0.0 | 0.0 | 0.0 |
| C6 Sat + Insat | 17.0 | 11.8 | 12.9 | 12.4 |
| C7 Sat + Insat | 2.3 | 3.2 | 3.7 | 3.5 |
| C8 Sat + Insat | 0.2 | 1.2 | 1.1 | 1.1 |
| C9 Sat + Insat | — | 0.0 | 0.0 | 0.0 |
| Benzene | 0.3 | 1.2 | 1.2 | 1.1 |
| Toluene | 0.1 | 2.9 | 2.8 | 3.0 |
| Ethyl-Benzene | — | 0.3 | 0.3 | 0.3 |
| p and m-Xylene | — | 2.1 | 2.2 | 2.2 |
| o-Xylene | 0.2 | 0.7 | 0.7 | 0.7 |
| 1,2,3 Trimethylbenzene | — | 0.1 | 0.1 | 0.0 |
| 1,2,4 Trimethylbenzene | — | 0.4 | 0.3 | 0.3 |
| 1,2,3 Trimethylbenzene | — | 0.2 | 0.2 | 0.2 |
| Others | — | 1.0 | 0.9 | 0.8 |
| Totals Olefins (%) (C2-C4) | — | 16.1 | 15.6 | 14.0 |
| LPG Fraction (%) | 16.9 | 24.0 | 22.4 | 20.5 |
| Total Aromatics (%) | 0.6 | 7.9 | 7.8 | 7.8 |
| Load Conversion (%) | — | 37.2 | 35.3 | 30.8 |

H = hours;
WHSV = 22.44 h⁻¹;
Reaction Temperature = 530° C.

TABLE 4

Composition (% Weight)

| Compounds | Feed | 2 H | 12 H | 24 H |
|---|---|---|---|---|
| Methane | — | 3.3 | 2.5 | 2.2 |
| Ethane | — | 6.7 | 5.2 | 4.6 |
| Ethene | — | 4.2 | 4.0 | 3.5 |
| Propane | — | 22.3 | 17.9 | 14.5 |
| Propene | — | 6.4 | 6.4 | 5.7 |
| Isobutane | 0.1 | 4.0 | 3.6 | 2.9 |
| n-Butane | 8.9 | 6.3 | 6.8 | 6.7 |
| trans-2-Butene | — | 0.9 | 0.9 | 0.9 |
| 1-Butene | — | 0.6 | 0.7 | 0.7 |
| Isobutane | | 1.5 | 1.6 | 1.5 |
| cis-2-Butene | — | 0.7 | 0.7 | 0.7 |
| Isopentane | 19.7 | 5.8 | 6.8 | 7.5 |
| n-Pentane | 29.8 | 3.0 | 4.1 | 4.9 |
| Pentanes | — | 0.1 | 0.1 | 0.1 |
| C6 Sat + Insat | 31.6 | 2.8 | 3.6 | 3.8 |
| C7 Sat + Insat | 8.1 | 0.6 | 1.3 | 1.3 |
| C8 Sat + Insat | 1.0 | 0.1 | 0.6 | 1.0 |
| C9 Sat + Insat | — | 0.0 | 0.1 | 1.2 |
| Benzene | 0.5 | 3.7. | 3.6 | 3.3 |
| Toluene | 0.3 | 12.1 | 11.5 | 10.4 |
| Ethyl-Benzene | — | 0.9 | 0.9 | 1.3 |
| p and m-Xylene | — | 7.5 | 7.9 | 9.8 |
| o-Xylene | — | 2.4 | 2.9 | 3.6 |
| 1,2,3 Trimethylbenzene | — | 0.4 | 0.5 | 0.7 |
| 1,2,4 Trimethylbenzene | — | 0.7 | 1.1 | 1.7 |
| 1,2,3 Trimethylbenzene | — | 0.5 | 0.8 | 1.1 |
| Others | — | 2.5 | 3.9 | 4.4 |
| Totals Olefins (%) (C2-C4) | — | 14.3 | 14.3 | 13.0 |
| LPG Fraction (%) | 9.0 | 32.6 | 28.3 | 24.1 |
| Total Aromatics (%) | 0.8 | 28.2 | 29.2 | 31.9 |
| Load Conversion (%) | — | 77.1 | 73.4 | 71.8 |

H = hours;
WHSV = 5.25 h⁻¹;
Reaction Temperature = 530° C.

TABLE 5

| | Conversion (% weight) | | |
|---|---|---|---|
| | 2 H | 12 H | 24 H |
| | 11.5 | 10.9 | 9.4 |
| Products | Selectivity (% Weight) | | |
| Methane | 2.4 | 2.1 | 2.2 |
| Ethane | 6.1 | 5.4 | 5.6 |
| Ethene | 11.8 | 11.5 | 11., 7 |
| Propane | 22.7 | 20.6 | 19.3 |
| Propene | 27.2 | 27.4 | 29.5 |
| Isobutane | 4.5 | 2.9 | 2.2 |
| trans-2-Butene | 4.8 | 4.9 | 5.2 |
| 1-Butene | 3.5 | 3.6 | 3.8 |
| Isobutane | 8.2 | 8.4 | 8.9 |
| cis-2-Butene | 3.6 | 3.7 | 4.0 |
| Benzene | 0.9 | 1.2 | 0.9 |
| Toluene | 4.2 | 4.0 | 3.9 |
| Ethyl-Benzene | 0.1 | 0.6 | 0.5 |
| p and m-Xylene | — | 3.0 | 1.9 |
| o-Xylene | — | 0.7 | 0.4 |
| 1,2,3 Trimethylbenzene | — | — | — |
| 1,2,4 Trimethylbenzene | — | | — |
| 1,3,6 Trimethylbenzene | — | — | — |
| Others | — | | |
| Total Olefins (%) | 59.1 | 59.5 | 63.1 |
| LPG Fraction (%) | 27.2 | 23.5 | 21.5 |
| Total Aromatics (%) | 5.2 | 9.5 | 7.6 |

H = hours;
WHSV = 85.1 h⁻¹;
Reaction Temperature = 530° C.

TABLE 6

| | Conversion (% weight) | | |
|---|---|---|---|
| | 2 H | 12 H | 24 H |
| | 37.2 | 35.3 | 30.8 |
| Products | Selectivity (% Weight) | | |
| Methane | 2.7 | 2.7 | 2.6 |
| Ethane | 6.6 | 6.8 | 6.5 |
| Ethene | 9.2 | 9.3 | 9.5 |
| Propane | 26.0 | 24.7 | 21.7 |
| Propene | 17.5 | 17.9 | 18.5 |
| Isobutane | 5.7 | 5.0 | 4.1 |
| trans-2-Butene | 3.0 | 3.0 | 3.2 |
| 1-Butene | 2.2 | 2.2 | 2.4 |
| Isobutane | 5.1 | 5.2 | 5.5 |
| cis-2-Butene | 2.2 | 2.3 | 2.4 |
| Benzene | 2.1 | 2.4 | 2.3 |
| Toluene | 6.7 | 6.9 | 8.6 |
| Ethyl-Benzene | 0.7 | 0.9 | 1.0. |
| p and m-Xylene | 5.1 | 5.6 | 6.5 |
| o-Xylene | 1.1 | 1.2 | 1.3 |
| 1,2,3 Trimethylbenzene | 0.3 | 0.2 | 0.0 |
| 1,2,4 Trimethylbenzene | 0.9 | 0.9 | 1.0 |
| 1,2,3 Trimethylbenzene | 0.6 | 0.6 | 0.7 |
| Others | 2.3 | 2.2 | 2.2 |
| Total Olefins (%) | 39.2 | 39.9 | 41.5 |
| LPG Fraction (%) | 31.7 | 29.7 | 25.8 |
| Total Aromatics (%) | 17.1 | 18.7 | 21.3 |

H = hours;
WHSV = 22.44 h⁻¹;
Reaction Temperature = 530° C.

TABLE 7

Conversion (% weight)

| | 2 H | 12 H | 24 H |
|---|---|---|---|
| | 77.1 | 73.4 | 71.8 |
| Products | Selectivity (% Weight) | | |
| Methane | 4.1 | 3.3 | 3.0 |
| Ethane | 8.3 | 6.9 | 6.3 |
| Ethene | 5.3 | 5.3 | 4.8 |
| Propane | 27.7 | 23.6 | 19.9 |
| Propene: | 7.9 | 8.4 | 7.9 |
| Isobutane | 5.0 | 4.7 | 3.9 |
| trans-2-Butene | 1.1 | 1.3 | 1.2 |
| 1-Butene | 0.8 | 0.9 | 0.9 |
| Isobutane | 1.9 | 2.2 | 2.1 |
| cis-2-Butene | 0.8 | 1.0 | 0.9 |
| Benzene | 4.0 | 4.0 | 3.9 |
| Toluene | 14.6 | 14.8 | 13.9 |
| Ethyl-Benzene | 1.1 | 1.2 | 1.9 |
| p and m-Xylene | 9.3 | 10.4 | 13.5 |
| o-Xylene | 3.0 | 3.8 | 5.0 |
| 1,2,3 Trimethylbenzene | 0.5 | 0.6 | 1.0 |
| 1,2,4 Trimethylbenzene | 0.9 | 1.5 | 2.4 |
| 1,2,3 Trimethylbenzene | 0.6 | 1.0 | 1.5 |
| Others | 3.1 | 5.1 | 6.0 |
| Total Olefins (%) | 17.8 | 19.1 | 17.8 |
| LPG Fraction (%) | 32.7 | 28.3 | 23.8 |
| Total Aromatics (%) | 34.0 | 37.3 | 43.1 |

H = hours;
WHSV = 5.25 h⁻¹;
Reaction Temperature = 530° C.

The present invention deals with a catalytic cracking process for selective production of olefins, especially light olefins (C₂-C₄) and other petrochemical supplies, such as, the LPG fraction (propane+butanes) and aromatics from condensed natural gas.

The products obtained from catalytic cracking of the fraction derived from condensed natural gas were analyzed with composition values (% weight) and included, preferably, between 6% and 16% for total olefins, from 11% to 33% for the LPG fraction and from 1% to 32% for the aromatics.

Although the invention's illustrative examples use composition values in % weight of total olefins, LPG fraction and aromatics mentioned above, experts in the field will easily understand that these ranges may (according to different processing conditions) extend, with no problems, to include percentage values by weight, respectively, of between 2-50, preferably between 4-30, and even more preferably between 6-20 for total olefins, between 5-70, preferably, 7-50, and still more preferably, 9-40, for the LPG fraction, and between 1-50, preferably, 1-40, and still more preferably, between 1-35, for the aromatics, without altering the scope of the present invention.

The processing data allows verification that the production of olefins is favored under conditions of greater space velocities, whereas the production of the LPG fraction and aromatics is favored under lower space velocity conditions. Lower space velocities favor the production of aromatics in the process, principally toluene.

The composition (% weight) of toluene falls within a preferable range of between 33% and 44% of the total aromatic fraction composition. Calculated selectivity values (% weight) of the effluent in tests 1, 2, and 3, include values ranging preferably between 17% and 63% for total olefins, from 21% to 33% for the LPG fraction and from 4% and 43% for the aromatics.

In a way similar to the experimental values found for the percentage in weight of products, the selectivity values (in percentages in weight) may also be extended to greater ranges, depending on the processing conditions used. In the same way, experts may understand that selectivity ranges, (always in percentages by weight) may be between 10-80, preferably between 15-70, and even more preferably between 17-65, for total olefins, between 5-50, preferably between 10-40, and even more preferably between 15-35, for the LPG fraction, and between 1-70, preferably between 3-60, and even more preferably between 5-45 for the aromatics.

1.-11. (canceled)

12. Selective catalytic cracking process of condensed natural gas liquid fraction to light olefins and other products, characterized in that it includes placing said liquid fraction of natural gas, rich in C5+ paraffins, in contact (within a reaction zone) with an MFI type zeolitic catalyst in acid form, having a pore size of at least 4 Angstroms, a silica/alumina ratio of between 20 and 100, and a grain size distribution between 0.510 mm and 0.044 mm, and where the processing conditions involve a temperature of between 350° C. and 650° C., space velocity of between 2 and 100h⁻¹ and atmospheric pressure, and afterwards carrying out the catalytic cracking to separate products, to recover a product enriched with light olefins, LPG fractions, and aromatics, and where the production of olefins is favored in conditions of higher space velocities, while the production of LPG fractions and aromatics are favored in conditions of lowered space velocities.

13. Process in accordance with claim 12, characterized by the use of an MFI type zeolitic catalyst in the form of dust, pellet or any other configuration.

14. Process in accordance with claim 12, characterized in that additional to before the use of said catalyst, it is pretreated at a temperature within the range of 200-650° C. for a period of 2 hours under the continuous flow of a carrier gas (preferably air or nitrogen).

15. Process in accordance with claim 12, characterized in that the MFI type catalyst is a ZSM-5 zeolite.

16. Process in accordance with claim 12, characterized in that the results obtained of total olefins (ethene+propene+butenes) in the effluent with a composition (% weight) falls within a range of between 2% and 50%

17. Process in accordance with claim 12, characterized in that the results of the LPG fraction in the effluent with a composition (% weight) fall within a range of between 5% and 70%.

18. Process in accordance with claim 12, characterized in that the results of the aromatic fraction in the effluent with a composition (% weight) fall within a range of between 1% and 50%.

19. Process in accordance with claim 18, characterized in that the composition (% weight) of toluene falls within a preferable range of between 33% and 44% of the total aromatic fraction composition.

20. Process in accordance with claim 12, characterized in that the results of selectivity (% weight) for total olefins (ethene+propene+butenes) fall within a range of between 10% and 80%.

21. Process in accordance with claim 12, characterized in that the results of selectivity (% weight) for the LPG fraction fall within a range of between 5% and 50%.

22. Process in accordance with claim 12, characterized in that the results of selectivity (% weight) for the aromatics fall within a range of between 1% and 70%.

23. Process in accordance with claim 12, characterized in that additional to before the use of said catalyst, it is pretreated at a temperature within the range of 300-600° C. for a period of 2 hours under the continuous flow of a carrier gas (preferably air or nitrogen).

24. Process in accordance with claim 12, characterized in that the results obtained of total olefins (ethene+propene+butenes) in the effluent with a composition (% weight) falls within a range of between 4% and 30%.

25. Process in accordance with claim 12, characterized in that the results obtained of total olefins (ethene+propene+butenes) in the effluent with a composition (% weight) falls within a range of between 6% and 20%.

26. Process in accordance with claim 12, characterized in that the results of the LPG fraction in the effluent with a composition (% weight) fall within a range of between 7% and 50%.

27. Process in accordance with claim 12, characterized in that the results of the LPG fraction in the effluent with a composition (% weight) fall within a range of between 9% and 40%.

28. Process in accordance with claim 12, characterized in that the results of the aromatic fraction in the effluent with a composition (% weight) fall within a range of between 1% and 40%.

29. Process in accordance with claim 12, characterized in that the results of the aromatic fraction in the effluent with a composition (% weight) fall within a range of between 1% and 30%.

30. Process in accordance with claim 12, characterized in that the results of selectivity (% weight) for total olefins (ethene+propene+butenes) fall within a range of between 15% and 70%.

31. Process in accordance with claim 12, characterized in that the results of selectivity (% weight) for total olefins (ethene+propene+butenes) fall within a range of between 17% and 65%.

32. Process in accordance with claim 12, characterized in that the results of selectivity (% weight) for the LPG fraction fall within a range of between 10% and 40%.

33. Process in accordance with claim 12, characterized in that the results of selectivity (% weight) for the LPG fraction fall within a range of between 15% and 35%.

34. Process in accordance with claim 12, characterized in that the results of selectivity (% weight) for the aromatics fall within a range of between 3% and 60%.

35. Process in accordance with claim 12, characterized in that the results of selectivity (% weight) for the aromatics fall within a range of between 5% and 45%.

36. Process in accordance with claim 12, wherein the MFI type zeolitic catalyst in acid form has a grain size distribution between 0.247 mm and 0.091 mm.
